(19) United States
(12) Patent Application Publication
(10) Pub. No.: US 2012/0183965 A1
(43) Pub. Date: Jul. 19, 2012

(54) NUCLEIC ACID DETECTION

(76) Inventors: David Ward, Guisborough (GB); David Edge, Warlingham (GB); Nelson Nazareth, Upper Dean (GB)

(21) Appl. No.: 13/261,083

(22) PCT Filed: Jun. 14, 2010
(86) PCT No.: PCT/GB2010/001156

§ 371 Date: Apr. 2, 2012

(30) Foreign Application Priority Data
Jun. 15, 2009 (GB) 0910297.1
Jul. 24, 2009 (GB) 0912894.3

(51) Int. Cl. G01N 21/64; C12M 1/40; G01N 21/17

(52) U.S. Cl. 435/6.12; 435/287.2

(57) ABSTRACT

Apparatus for detecting a plurality of analytes in a sample and comprising:
a reaction vessel;
means for subjecting a sample in the reaction vessel to a multiplication process;
a separation stage operable to separate amplimer constituents according to size;
optical detection means for quantifying the sizes present and determining the colour of each size; and
means for comparing the resulting quantification and colours with known data to determine the nature of the or each or a proportion of the or each target amplimers present.

Processes employing the apparatus are also described.

### FIELD OF THE INVENTION

The present invention relates to the detection and identification of nucleic acids and in particular to a multiplex nucleic acid detection system.

### BACKGROUND TO THE INVENTION

Multiplex detection of nucleic acids, for example of viruses, is described inter alia in Patent Specification WO/2004/085455 which describes samples being placed in a plurality of wells and then each subjected to the detection process in parallel.

Multiplex detection of disease causing agents has been shown possible by immunological techniques, one example being the LUMINEX system described in patent specification WO/2002/024959. Another technique is the hybridisation of target molecules to an array as described in patent specification WO/2008/054830. A disadvantage of such approaches is the time taken to generate the results. Furthermore they require a large sample size to ensure that the often very dilute analyte can be successfully detected. An alternative approach has been the PCR process described in U.S. Pat. No. 4,683,202. Traditionally the nucleic acid was detected by size fractionation following PCR, a time consuming process, but having the possibility to multiplex analytes by their size, when subsequently subjecting the generated amplimers to some downstream process such as electrophoresis. Latterly, the majority of nucleic acid detection systems rely on real-time PCR such as is described in U.S. Pat. No. 6,814,934. A distinct disadvantage of real-time PCR is a physical limitation on the instruments utilised to perform this testing—they are limited to detecting up to 5 target molecules. This limitation is physical, by virtue of the design of their optical systems and further, the optical design leads to the problem of cross talk between optical channels. This in effect makes it very difficult to quantify the levels of analyte present due to light from the desired analyte being overshadowed by light from other products present in the reaction vessel.

The objective remains therefore of being able to detect all the pathogens in a single sample in as short a time period as possible. An outbreak of an unidentified infection, when the causative agent could be one of any of a group of viruses and/or bacteria, presents well the case for fulfillment of this need. This objective is also true for a wide range of fields, such as human genetics and DNA fingerprinting for scene of crime analyses. A further important objective is the accurate detection and identification of short nucleic acid sequences, in the range of sub 75 but preferably sub 60 bp amplicon size. These cannot be reliably analysed with conventional approaches, including those above-mentioned.

The limitations placed upon the currently available chemistries are imposed by the specifics of the testing methodologies themselves. Generic methods of detection including the use of intercalating dyes such as SYBR, allow detection and yet have the major disadvantage in the field of application, in that they are not nucleic acid sequence specific. This has led to the invention of a number of detection systems, or probes, that require a molecular interaction, requiring that the correct nucleic acid sequence be present in order for a signal to be generated. One example of these is described in patent specification WO97/46707. The disadvantage to this approach is that the extra molecule places a design burden on such techniques, in that there is a minimum size amplicon that may be detected. Further, the molecular detection provided by the probe is a higher order event, in that time must be allowed in order for the signal to be generated.

Self-probing primers are known in the art, however those such as the LUX system are not sequence specific, and any spurious product generated will similarly generate a signal. Other known chemistries, namely Scorpions GB 2338301 and the ‘Angler’ approach Lee, M. A. et al. (2002), Analytica Clinica Acta 457: 61:70; Whitcombe, D. et al. (1999), Nature Biotechnology 17: 804-807, utilise priming sequences that themselves probe within the amplified sequence. These therefore require a higher order event to take place, namely the hybridisation to the desired target and further do not cater for short amplimers, since sufficient room must be allowed for the sequence to be probed to be contained within the amplimers.

The present invention proposes a number of means of overcoming these limitations, with particular reference to detection of short nucleic acid fragments below the 60 bases in size required by existing chemistries. Intercalating dye approaches will tend to appear to possess low specificity with short products, even though amplification efficiency is high, as the high background masks the relatively low signal generated by shorter amplicons. Additionally, such short amplicons simply do not possess enough available bases for traditional probe methods to be employed and around 60 bases appears to be the cut-off point.

Such short amplicons are advantageous in tests where the target DNA may be sheared, such as is the case when attempting to detect pathogen nucleic acids that have been processed by the host immune system. Alternatively, small interfering RNA (siRNA) research focuses on double stranded RNA molecules ranging in size from 20 to over 30 bases and detection of these molecules is not possible using existing probe technologies. It can be the case that DNA sequences differentiating pathotypes can consist of short repeats of nucleotides and, being able to accurately determine the numbers of repeats only and without interference from identical genomic regions, can be advantageous. An additional benefit of such an approach to all assay types is an increased reaction speed and PCR efficiency inherent in such short amplicons.

In this patent specification the word vessel refers to any device capable of holding a substance or a sample to be processed and may accordingly comprise or consist of a well, a tube (open or closed) a slide, perhaps in the form of a silicon chip or a tray. The invention is particularly concerned with microtitre vessels in well form.

In this patent specification the term thermal cycling is used to refer to heating the sample cyclically to a plurality of temperatures. A typical thermal cycling process is polymerase chain reaction (PCR) when three temperatures—the upper denaturing temperature, the intermediate, extension temperature and the lower, recombination temperature, are employed. Ideally during a thermal cycling process the required temperatures are reached and maintained as accurately and rapidly as possible in order to minimize the time taken by the process.

There are systems that perform real-time analyses. Patent Specification WO2004045772, describes one such. However these systems are limited to multiplex analysis methods such as melting point determination and above mentioned real-time PCR process. Likewise there are offline systems for size separation such as the DNA sequencer described in U.S. Pat. No. 5,552,322.

An important object of the present invention is, therefore, to provide a system for efficient multiplex analysis of nucleic acid analytes.

### SUMMARY OF THE INVENTION

According to a first aspect of the invention a method of detecting a plurality of analytes in a sample comprises:
selecting one or more primers in accordance with the expected nature of the analyte, labelling each primer with a different dye and placing same in a reaction vessel;
placing a sample to be analysed in said reaction vessel;
subjecting said sample to a multiplication process;
separating the constituents of the multiplied sample, hereinafter called the amplimer, according to size;
quantifying the sizes present and determining the colour of each size; and
comparing the resulting quantification and colours with known data to determine the nature of the or each or a proportion of the or each target amplimers present.

It will be appreciated that the multiplication process of this aspect of the invention may be effected in a single reaction vessel, notwithstanding the multiplicity of nucleic acids that may be present in the sample.

The separation of the amplimer constituents may be carried out by subjecting the amplimer to a voltage in order to perform electrophoresis. Alternatively centrifugation may be employed.

Typical separation media include agarose, polyacrylamide and others well known in the art.

The process may include transferring the multiplied sample from the reaction vessel to an optical detection apparatus, the latter perhaps comprising a capillary tube, wherein the quantification of sizes and colour determination occur. The transer may be effected using a micro fluidics system. It will however be appreciated that the whole process may take place within a kit which could in one embodiment be of hand held dimensions, in another of small bench-top dimensions and in another of laboratory equipment proportions, and in each case automated.

Despite the possible separation of the multiplication apparatus from the optical detection apparatus it is a feature of the invention that it is readily possible to provide apparatus for effecting the above process which is manually portable and hence susceptible of field use, for example by a veterinary going to visit a farm where there has been an outbreak of some disease or other. The signs of disease from the animal or herd concerned will provide some indication as to the nature of the causative pathogen and the primers and dyes can be selected accordingly.

The facility can be capable of detecting at least two analytes, though over ten, even over twenty should be achievable in a single closed tube assay.

According to a second aspect of the invention there is provided apparatus for detecting a plurality of analytes in a sample and comprising:
a reaction vessel;
means for subjecting a sample in the reaction vessel to a multiplication process;
a separation stage operable to separate amplimer constituents according to size;
optical detection means for quantifying the sizes present and determining the colour of each size; and
means for comparing the resulting quantification and colours with known data to determine the nature of the or each or a proportion of the or each target amplimers present.

Preferably the reaction vessel is a microtitre well, the sample multiplication means comprises a PCR apparatus, the separation stage comprises means for applying a voltage to the amplimer and the means for subjecting the amplimer to a separation voltage comprises electrophoresis apparatus, the optical detection means comprises a spectral detector.

The present invention thus provides a facility for simultaneously detecting a plurality of nucleic acids by multiplying them to generate amplimers of the expected nucleic acids, for example by PCR, then separating the generated amplimers by physical size. Effectively this means concurrently being able to identify the amplified targets by size as well as colour. This facility greatly increases the number of analytes, in this case pathogens, detectable in one operation. Moreover the operation can be completed within 60, possibly even 30 minutes.

According to a feature of the invention oligonucleotides in the sample are fluorescently labelled within the facility. In this way quite short oligonucleotide primer sequences can be detected. Suitable dyes include, inter alia fluorescent labels such as fluorescein, TET, HEX. This is a so-called chemistry portion of the system.

This chemistry portion has the advantage over prior art systems of removing the requirement for an additional molecule, formerly necessary to generate a sequence specific signal. This brings an important twofold advantage over existing methods; one, a smaller area can be considered for sequence detection and two, the smaller amplicons and lack of higher order event, enable more rapid detection of the pathogen of interest.

The facility may accordingly be used to identify PCR products differing in size by 1 bp, with fifteen or more being identified simultaneously using the fluorescently labelled primers described above.

The products are then separated using the separation system as outlined above. Then when the separated fragments are subjected to optical interrogation by a spectroscopic analyser suitable software can be employed to interpret the results for the end user automatically, based on the presence or absence of light within the expected wavelength and size bandings.

The invention is thus a system capable of rapid multiplex real-time PCR and subsequent separation by size of the resultant amplicons, the identities of which can be automatically resolved by specifically written software. Moreover the system can be used to discriminate any short (sub 60 bp) amplimers for a variety of targets obvious to those operating in this field.

In operation it is envisaged that the tests can be constructed so that all targets of a given group will be labelled with an identical dye and real-time analysis performed to identify the group. Sub-typing within the group can then be immediately provided by the subsequent size separation. This is assay dependent and the system can then discriminate between minimally one and maximally one hundred analytes in a single vessel (this being ten differing colours and ten varying amplicon sizes). This system component of the invention has three stages, examples of which will now be outlined below.

The first stage advantageously comprises a rapid and accurate thermal cycling system as outlined in patent specification WO 2008107683. This stage can be completed in under 30 minutes.

The multiplex detection system may feature one or more illumination sources, including single or multiple illumination sources. These will preferably be diode pumped solid state laser or lasers, but could be any one of LEDs, gas pumped lasers, lamps etc. In another envisaged embodiment a series of such illumination sources may be used so as to further increase the range of dyes that the system can utilise.

The invention advantageously makes use of a spectral detector having minimally 6 nm resolution, but preferably 1 nm. This allows discrimination of twelve dyes and as such allows detection of significantly more amplimers than existing technology, which is currently limited to five agents. The wavelengths to be analysed would minimally be 500-700 nm, but preferably 300-1000 nm. Such a system can be capable, by means of dye deconvolution software, of discriminating between dyes differing by only 3 nM in peak fluorescence emission. Deconvolution software can accurately separate the individual spectra from each of the analytes present.

Each of the envisaged probe systems can take advantage of recent advances in the use of modified bases, for example LNA molecules or any modified base known in the art. The advantage of such molecules is that using the described probe systems it has been possible to detect amplimers as short as twenty bases in length.

Other envisaged embodiments of this invention rely on the placement of the two fluorescent dyes placed separately on the forward and reverse primers. Only in the correct amplified product will these fluorescent molecules be spatially close enough for the FRET to take place and hence a signal be generated.

The step of comparing the resulting quantification and colours with known data to determine the nature of the or each or a proportion of the or each target amplimers present may be effected using suitable bespoke deconvolution software. The software will automatically interpret the results for the end user, based on the presence or absence of light within the expected wavelength bandings.

As the optical system may be arranged to allow the throughput of the nucleic acid assays from a reaction chamber so tests can be performed in a number of thermal cyclers concurrently with the resulting amplimers then transferred to a single reader, thus rapidly to determine the diagnostic outcome.

As to the form of PCR which might be employed any of the currently accepted real-time PCR detection chemistries are possible. The probe system may take the form of ResonSense™ probes as described in Patent Specification WO/1999/028500. However the chemistries herein described are preferred.

### SPECIFIC EMBODIMENTS

The invention will now be particularly described with reference to the accompanying drawings, of which:

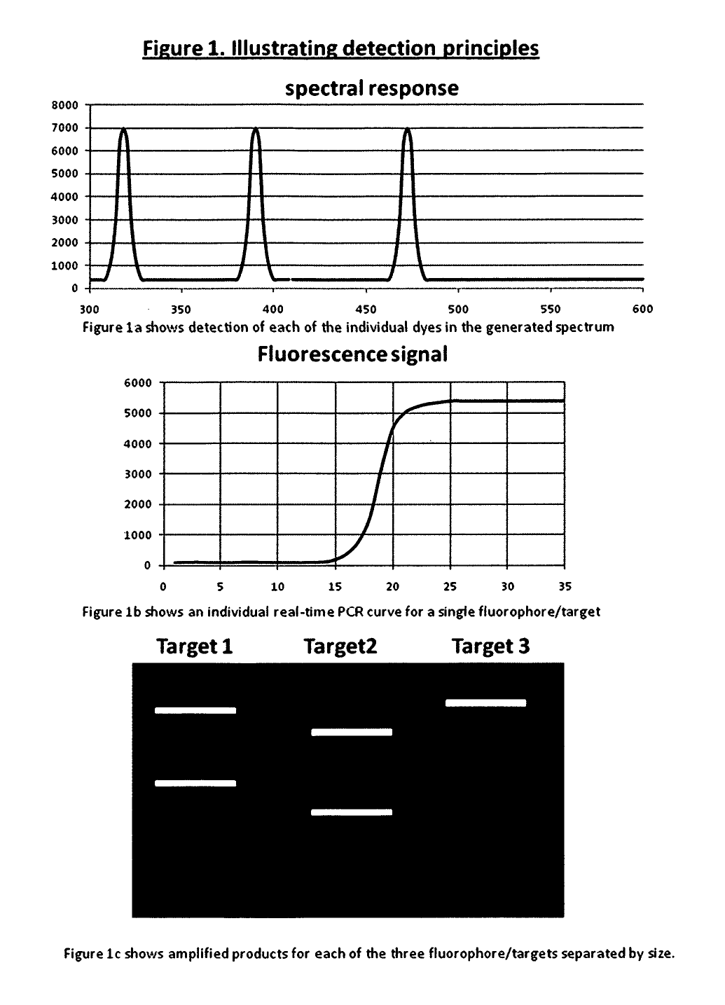

FIG. 1 (1a, 1b, 1c) illustrate the detection principles of the invention;

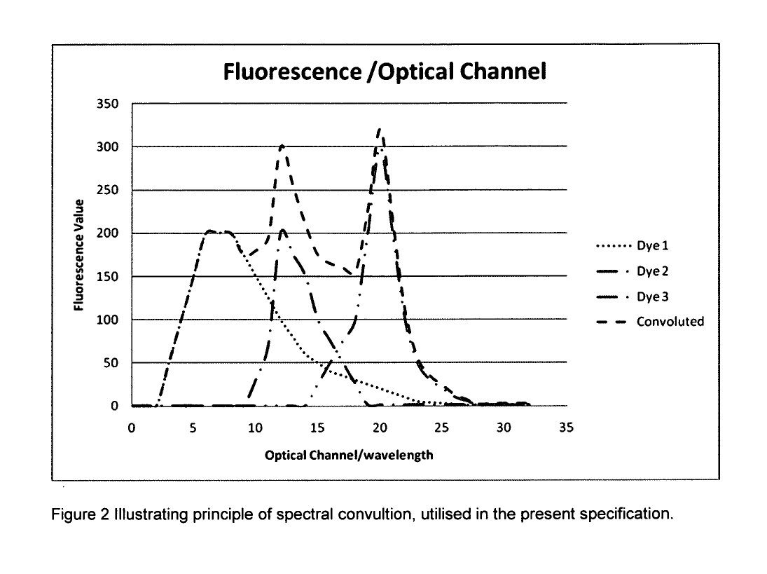

FIGS. 2, 3 and 4 illustrate embodiments of the chemistry phase of the invention;

FIG. 5 shows the interaction between fluorescent peaks;

FIG. 6 illustrates the separation of the amplimer constituents with time; and

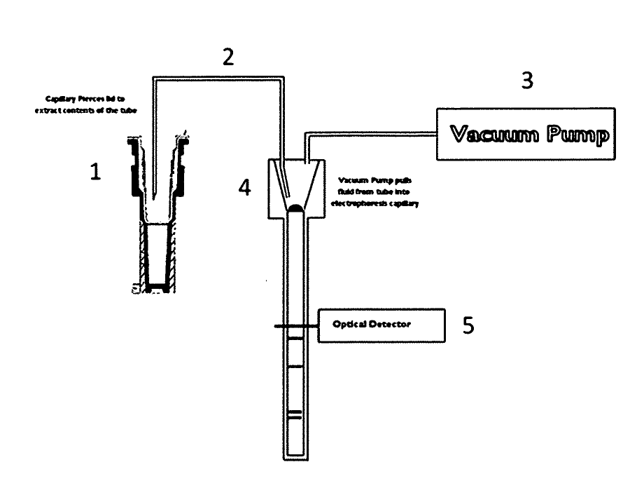

FIGS. 7 to 10 are schematic diagrams of an apparatus in accordance with the invention.

The first embodiment of the invention employs a sequence specific primer labelled at the 5 prime end with a fluorescent dye, preferably a longer wavelength dye such as CY5. The probing system binds to its intended target and is then available for priming the amplification of the desired sequence. The labelled primer is amplified in a reaction mixture containing an intercalating dye, with SYBR being preferred, although other suitable dyes such as SYTO 9 and EVA GREEN are known in the art. Ideally this dye is at a shorter wavelength than the dye tagged to the 5 prime end of the primer molecule. Upon illumination with a light source, a blue laser in the preferred embodiment, the SYBR dye will fluoresce and transfer its light energy to the CY5 by the FRET principle, the laser itself being unable to excite the CY5 dye. The CY5 signal is then detected and used to measure the progression of the biological reaction and permit detection of the potential pathogen at a molecular level. This approach is outlined in FIGS. 2 to 4.

The second preferred embodiment takes the form of a scanner and does not therefore require a thermal cycling system. Such a scanner can allow the throughput of the nucleic acid assays to be increased, as the tests could be performed in a number of thermal cyclers concurrently and then transferred to a single reader, to rapidly determine the diagnostic outcome. This method would be suitable for separation by colour only and as such would detect minimally one but readily up to twelve different targets in a single vessel.

A third embodiment is the incorporation of a tail sequence into the primer itself. The presence of a tail molecule allows for a sequence specific probe chemistry, such as Taqman to be used on such short amplicons, which would be physically impossible without such a modification having been made.

A fourth embodiment may be the use of fluorescent dye terminators into the amplification product. If the primers are CY5 labelled and constructed in an overlapping conformation only a single base extension is required, both to complete the biological reaction and to generate the fluorescent signal

As can be seen in FIG. 5 although three separate optical “peaks” may be present in the return spectrum from the sample each “peak” is amalgamated into a spectrum. This creates “cross talk” between “peaks” which must be interpreted and re separated by the deconvolution software which understands how the dyes respond when excited and understands the peak shapes individually as well as how the presence of one “peak” will provide secondary excitation to dyes in the same “sample”. As an example Dye 1 is excited by the LASER, This generates a fluorescence signal which excites dye 2 along with the LASER source, creating a different optical signal from Dye 2 than if only the LASER was solely performing the excitation

The preferred embodiment further increases the number of molecules that can be discriminated, by firstly performing a real-time PCR reaction, capable of multiplex detection and subsequently separating the amplified molecules by size using electrophoresis.

FIGS. 7 and 8 show a single use reaction vessel comprising a thermal cycling chamber and an additional channel containing a fractionating substance such as agarose, polyacrylamide or any other known in the art and suitable for electrophoretic separation to occur. The vessel is subjected to a thermal cycling reaction and in doing so generates intended PCR products in the presence of the correct analytes and thus generates fluorescent signals detected by the system. Following the completion of the reaction a micro fluidics system transfers a portion of the sample to the co-located separation medium and be ordered by size by the process of electrophoresis.

The preferred embodiment enables resolution of Single Nucleotide Polymorphisms (SNPs) by the labelling of each variation with differing dyes. In this way the SNP may be detected by either time taken to pass the optical detector or indeed by the wavelength of emitted light. Voltages to the electrode in the electrophoresis medium may be adjusted such that a high voltage is applied while the sample is “hunted for” (i.e. no fluorescence is detected) then reduced to increase the resolution of the detection and SNP analysis in the size ranges in which the amplicons are expected to fall, thus reducing time to detection while maintaining accuracy and resolution.

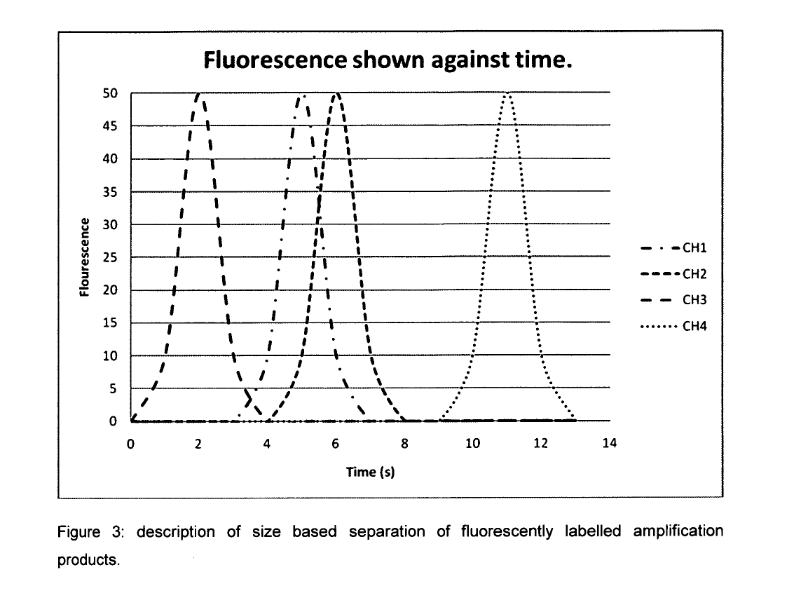

FIG. 3 shows four colours detecting four individual polymorphisms. It can be seen that the SNPs labelled in red and blue respectively would not be able to be resolved accurately by conventional size separation techniques. The dyes bound to the amplimers make the resolution of these comigrating species much more straightforward. This factor increases the amount of products that can be resolved as the proposed system can separate by both size and wavelength of emitted light.

The size fractionated sample is then subjected to spectral evaluation by the above described spectroscopic analyser. This system allows the detection of minimally two but actually well over twenty agents in a single assay

As shown in FIG. 7 a reaction vessel (1) has a reaction section wherein the PCR occurs. The required heating and cooling is effected by a peltier effect module (4) associated with a heat source/sink 6 and subject to reversing voltage polarity. Thermal energy is transferred to the reaction vessel through a thermally conductive reaction vessel holder (3). Below this reaction chamber is a capillary 11 containing electrophoresis gel. Shown on the capillary are a pair of electrical contacts (2) whereon a bias voltage may be supplied via electrical contacts (5) in order to facilitate the labelled DNA to traverse the gel towards the selected electrode.

LASER(s) excitation (7) is passed through the electrophoresis gel generating a fluorescence signature (8) when a labelled nucleic acid passes in front of the LASER(s).

This fluorescence signature (8) is directed over a diffraction grating (9) generating a spectrum across detector (10)

The capillary passes through a heat source/sink 6 which is held at a constant temperature of the order of 40-60° C. removal module (HRM) that allows the electrophoresis to occur at a higher voltage than would normally be allowed due to the generated heat this higher voltage creates.

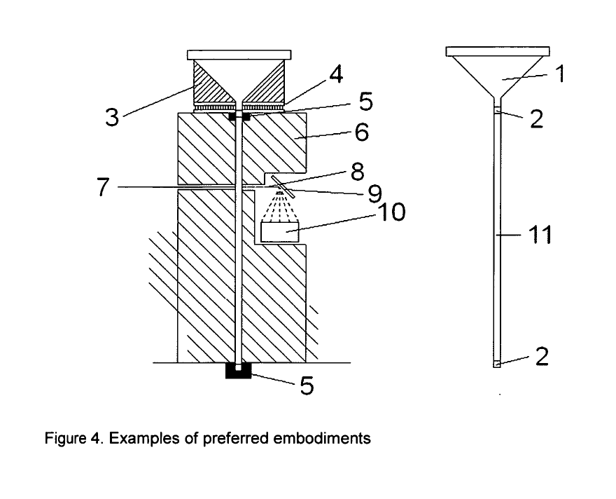

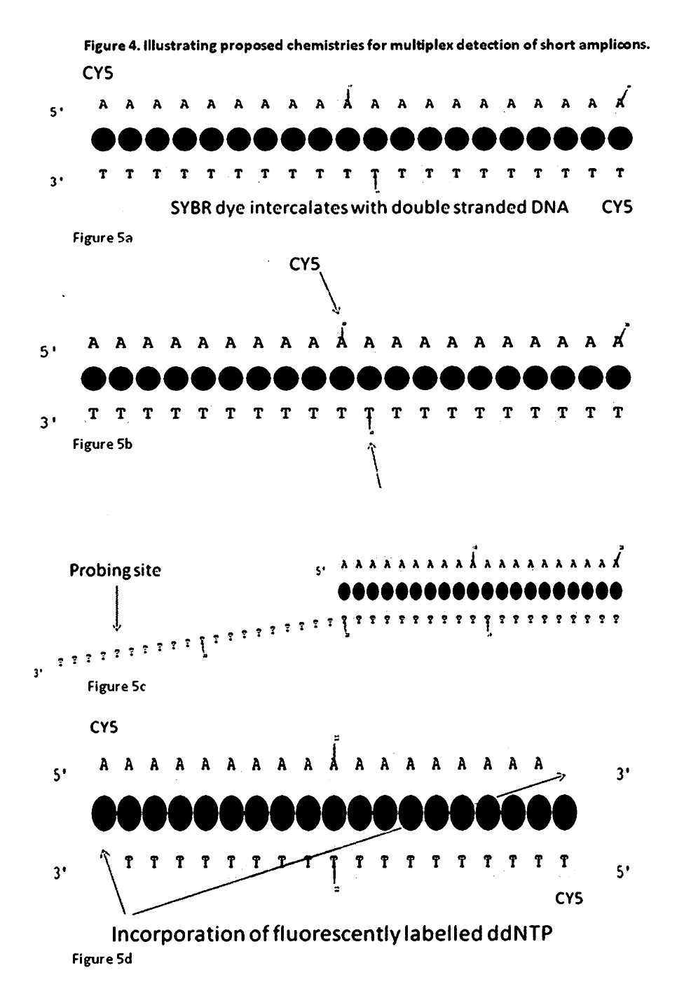

In order to complete these assays a chemistry suitable for amplification and detection of these short nucleic acid targets is required. The first envisaged embodiment of the invention is a sequence specific primer labelled at the 5 prime end with a fluorescent dye, preferably longer wavelength dye such as CY5, but not limited and including all dyes known in the art. The probing system will bind to its intended target and be available for priming the amplification of the desired sequence. The labelled primer is amplified in a reaction mixture containing an intercalating dye, with SYBR being the preferred embodiment, although other suitable dyes such as SYTO 9 and EVA GREEN are known in the art. Crucially this dye is to be at a shorter wavelength than the dye tagged to the 5 prime end of the primer molecule. Upon illumination with a light source, a blue laser in the preferred embodiment, the SYBR dye will fluoresce and transfer its light energy to the CY5 by the FRET principle, the laser itself being unable to excite the CY5 dye. The CY5 signal is then detected and used to measure the progress of the biological reaction and permit detection of the potential pathogen at a molecular level. This approach is outlined in FIG. 4.

Each of the envisaged probe systems can take advantage of recent advances in the use of modified bases, for example LNA molecules or any modified base known in the art. The advantage of such molecules is that using the described probe systems it has been possible to detect amplimers as short as 20 bases in length.

Other envisaged embodiments of this invention rely on the placement of the two fluorescent dyes placed separately on the forward and reverse primers. Only in the correct amplified product will these fluorescent molecules be spatially close enough for the FRET to take place and hence a signal be generated as in FIG. 4b.

A third embodiment is the incorporation of a tail sequence into the primer itself. The presence of a tail molecule allows for a sequence specific probe chemistry, such as Taqman to be used on such short amplicons, which would be physically impossible without such a modification having been made. This is highlighted in FIG. 4c, this approach has previously been utilised to provide complimentary sequence for hairpin generation or to provide a binding site to be subsequently used in a self-probing amplicons arrangement such as the angler chemistry. The use of a 5′ sequence tail to provide a target for probe hybridisation in short amplicons where a suitable target candidate could not otherwise be selected is novel in this regard.

A final potential embodiment is the use of fluorescent dye terminators into the amplification product. The primers being CY5 labelled and designed so in an overlapping conformation, such that only a single base extension is required, both to complete the biological reaction and to generate the fluorescent signal as in FIG. 4d.

It will be appreciated that whilst a kit as above described is capable of carrying out the whole process claimed and described herein it is also capable of carrying out a detection process where it is known that at most say four or five different nucleic acids are present, omitting the chemistry phase of the process altogether.

FIG. 8 represents a further embodiment of the invention. In that embodiment an extant real-time PCR arranged so that the completed amplimer may be collected for subsequent size separation. The reaction vessel is supplied with a pierceable lid and following completion of the real-time PCR multiplication process, the now fluorescently labelled products are subsequently separated by size. This apparatus incorporates a fluidics system capable of piercing the vessel lid and delivering the amplimer to a capillary system arranged for the application of electrophoresis to separate the fluorescently labelled products by size.
1 reaction vessel containing amplification products
2 fluid transfer capillary tubing
3 vacuum pump for moving fluid
4 Electrophoresis capillary
5 optical detector

It will be appreciated also that, whereas one might have employed say a 96 well array each containing some of the same sample, and submitting the whole array to a PCR process, the present invention permits the use of a single well, this reducing considerably the size of sample required in order successfully to analyse its constituent nucleic acids and also the size of the kit required for the process.

1. A method of detecting a plurality of analytes in a sample and comprising:
selecting one or more primers in accordance with the expected nature of the analyte, labelling each primer with a different dye and placing same in a reaction vessel;
placing a sample to be analysed in said reaction vessel;
subjecting said sample to a multiplication process;
separating the constituents of the multiplied sample, hereinafter called the amplimer, according to size;
quantifying the sizes present and determining the colour of each size; and
comparing the resulting quantification and colours with known data to determine the nature of the or each or a proportion of the or each target amplimers present.

2. A method as claimed in claim 1 and wherein the multiplication process is effected in a single reaction vessel.

3. A method as claimed in claim 1 or claim 2 and wherein the separation is effected by applying a voltage.

4. A method as claimed in claim 3 and wherein the separation means employs electrophoresis.

5. A method as claimed in any one of claims 1 to 4 and wherein the primers comprise agarose or polyacrylamide.

6. A method as claimed in any one of the preceding claims and comprising transferring the multiplied sample from the reaction vessel to an optical detection apparatus.

7. A method as claimed in claim 6 and wherein the transfer is effected with a micro-fluidics system.

8. A method as claimed in claim 6 and wherein the optical detection apparatus comprises a capillary tube.

9. A method as claimed in any one of the preceding claims and wherein the reaction vessel is a microtitre well.

10. A method as claimed in any one of the preceding claims and wherein the multiplication process comprises PCR.

11. A method as claimed in claim 10 and wherein the PCR process is as outlined in patent specification WO 2008107683.

12. A method as claimed in any one of the preceding claims and wherein quantifying the sizes present and determining the colour of each size comprises a multiplex optical detection system featuring one or more illumination sources.

13. A method as claimed in claim 12 and wherein the illumination sources comprise diode pumped solid state lasers.

14. A method as claimed in claim 12 or claim 13 and wherein the optical system employs a spectral detector having minimally 6 nm resolution

15. A method as claimed in claim 12 or claim 13 and wherein the optical system employs a spectral detector having 1 nm resolution.

16. A method as claimed in any one of claims 12 to 15 and wherein the wavelengths to be analysed are 500-700 nm.

17. A method as claimed in any one of claims 12 to 15 and wherein the wavelengths to be analysed are 300-1000 nm.

18. A method as claimed in any one of the preceding claims and wherein the step of comparing the resulting quantification and colours with known data to determine the nature of the or each or a proportion of the or each target amplimers present comprises the application of a deconvolution software, based on the presence or absence of light within the expected wavelength bandings.

19. A method as claimed in any one of the preceding claims and wherein labelling the primers includes labelling short oligonucleotide primer sequences with fluorescent dyes.

20. A method as claimed in claim 19 and wherein the dyes comprise one or more of fluorescein, TET, HEX.

21. Apparatus for carrying out a method as claimed in any one of the preceding claims and comprising:
a reaction vessel;
means for subjecting a sample in the reaction vessel to a multiplication process;
a separation stage operable to separate amplimer constituents according to size;
optical detection means for quantifying the sizes present and determining the colour of each size; and
means for comparing the resulting quantification and colours with known data to determine the nature of the or each or a proportion of the or each target amplimers present.

22. Apparatus as claimed in claim 21 and wherein the reaction vessel is a microtitre well.

23. Apparatus as claimed in claim 21 or claim 22 and wherein the sample multiplication means comprises a PCR apparatus.

24. Apparatus as claimed in any one of claims 21 to 23 and wherein the means for subjecting the amplimer to a separation voltage comprises electrophoresis apparatus.

25. Apparatus as claimed in any one of claims 21 to 24 and wherein the optical detection means comprises a spectral detector.

26. Apparatus as claimed in any one of claims 21 to 25 and wherein the separation stage comprises means for applying a voltage to the amplimer.

27. Apparatus as claimed in any one of claims 21 to 26 and which comprises a portable, hand holdable, kit.

28. A method of separating amplimers in a nucleic acid amplification process and employing electrophoresis.

29. A method as claimed in any one of claims 1 to 20 and 27 and substantially as hereinbefore described.

30. Apparatus as claimed in any one of claims 21 to 27 and substantially as hereinbefore described.
